(19) United States
(12) Patent Application Publication
(10) Pub. No.: US 2017/0356028 A1
(43) Pub. Date: Dec. 14, 2017

(54) BACTERIAL EPIGENOMIC ANALYSIS

(71) Applicant: Ibis Biosciences, Inc., Carlsbad, CA (US)

(72) Inventor: Stanley Motley, Carlsbad, CA (US)

(73) Assignee: bis Biosciences, Inc., Carlsbad, CA (US)

(21) Appl. No.: 15/521,211

(22) PCT Filed: Oct. 22, 2015
(86) PCT No.: PCT/US2015/056969

§ 371 Date: Apr. 21, 2017

Related U.S. Application Data
(60) Provisional application No. 62/067,232, filed on Oct. 22, 2014.

(51) Int. Cl. C12Q 1/68; G06F 19/18; G06F 19/00; C12Q 1/70; G06F 19/28

(52) U.S. Cl. CPC C12Q 1/689; C12Q 1/70; G06F 19/28; G06F 19/3493; G06F 19/18; C12Q 2600/154

(57) ABSTRACT

Provided herein are systems and methods for determining the epigenetic sequences and signatures of bacteria, methods of characterizing bacteria based thereon, and methods of use thereof.

### CROSS-REFERENCE TO RELATED APPLICATIONS

The present Application claims priority to U.S. Provisional Application Ser. No. 62/067,232 filed Oct. 22, 2014, the entirety of which is incorporated by reference herein.

### FIELD

Provided herein are systems and methods for determining the epigenetic sequences and signatures of microbes, methods of characterizing microbes (e.g., bacteria, viruses, etc.) based thereon, and methods of use thereof.

### BACKGROUND

DNA modification (e.g., methylation) controls many important pathways in microorganisms (e.g., including those involved in virulence mechanisms in pathogenic bacteria and viruses. Conventional DNA sequence analysis does not identify DNA engineering events that affect modification (e.g., methylation) status.

### SUMMARY

Provided herein are systems and methods for determining the epigenetic sequences and signatures of microorganisms, methods of characterizing microorganisms (e.g., bacteria and viruses based thereon, and methods of use thereof. In some embodiments, provided herein are methods), compositions, and kits for determining the epigenetic signature of microorganisms; and bioforensics, attribution, determination of virulence, and development of therapeutics and diagnostics based thereon.

In some embodiments, provided herein are methods of characterizing a microorganism (e.g., bacteria, virus, etc.) in a sample comprising: (a) sequencing nucleic acid from the microorganism, wherein said sequencing results in an epigenomic signature of said microorganism; (b) comparing the epigenomic signature to a reference; and (c) identifying characteristics of said microorganism based on similarities and/or differences between the epigenomic signature of said microorganism and the reference. In some embodiments, the reference correlates at least one microorganism characteristic with an epigenomic microorganism reference signature. In some embodiments, the reference correlates at least one microorganism characteristic (e.g., bacterial characteristic, viral characteristic, etc.) with a sub-genomic microbial reference signature. In some embodiments, the at least one microbial characteristic is selected from species, strain, sub-strain, serotype, virulence level, pathogenicity, origin, known geographical range, antibiotic resistance or sensitivity, and culture conditions. In some embodiments, the epigenomic signature is an epigenomic sequence. In some embodiments, the reference is a database of microbial (e.g., bacterial, viral, etc.) epigenetic signatures. In some embodiments, the reference is a database of epigenomic microbial epigenetic signatures. In some embodiments, the reference is a database of microbial epigenetic sequences. In some embodiments, the reference is a database of microbial epigenomic sequences. In some embodiments, comparing the epigenomic signature to a reference comprises querying the database for epigenomic signature matches. In some embodiments, comparing the epigenomic signature to a reference comprises querying the reference for sub-genomic epigenetic signature matches. In some embodiments, the sequencing is performed by a non-amplification sequencing technique. In some embodiments, the sequencing is performed by a single molecule sequencing technique. In some embodiments, the sequencing is performed by a massively-parallel sequencing technique. In some embodiments, methods comprise sending the epigenomic signature of said microbe to a third party to be characterized; and receiving a report identifying characteristics of said microbe. In some embodiments, sending and receiving are performed electronically.

In some embodiments, provided herein are methods of characterizing a microbial bioagent (e.g., virus, bacteria, etc.) comprising: (a) exposing (i) a single nucleic acid molecule from the bioagent and (ii) sequencing reagents to conditions that allow determination of the epigenetic sequence of the single nucleic acid molecule; (b) comparing the epigenetic sequence of the single nucleic acid molecule or a representation thereof to a reference; and (c) identifying characteristics of the microorganism based on similarities between epigenetic sequence of the single nucleic acid molecule or a representation thereof to a reference. In some embodiments, the single nucleic acid molecule is a fragment of a whole genome nucleic acid from the microorganism. In some embodiments, methods further comprise fragmenting the whole-genome nucleic acid from the microorganism. In some embodiments, methods (or steps thereof) are performed in parallel for multiple single nucleic acid molecules that are fragments of the whole-genome nucleic acid from the microorganism. In some embodiments, the epigenetic sequence or a representation thereof for each of the multiple single nucleic acid molecules are compared to the reference. In some embodiments, methods comprise identifying characteristics of the bacteria based on similarities between the epigenetic sequences or representations thereof of any of the multiple single nucleic acid molecules and the reference. In some embodiments, the multiple single nucleic acid molecules collectively comprise the entire whole-genome nucleic acid from the microorganism. In some embodiments, methods comprise generating an epigenomic sequence or an epigenomic signature from the epigenetic sequences of the multiple single nucleic acid molecules that are fragments of the whole-genome nucleic acid from the bacteria. In some embodiments, methods comprise comparing the epigenomic sequence or the epigenomic signature to the reference. In some embodiments, methods comprise identifying characteristics of the microorganism based on similarities between the epigenomic sequence or the epigenomic signature and the reference. In some embodiments, the reference is a database of epigenetic data of multiple different microorganisms. In some embodiments, the reference is a database of microorganism epigenetic sequences, epigenetic signatures, or other representations thereof. In some embodiments, the reference is a database of microorganism epigenomic sequences, epigenomic signatures, or other representations thereof. In some embodiments, the multiple different bacteria are: different species, different serotypes, different strains, different substrains, and/or grown under different conditions. In some embodiments, each entry of epigenetic data in the database is correlated or indexed to characteristics of the respective bacteria.

In some embodiments, provided herein are methods of responding to a microbial threat comprising: (a) obtaining (or receiving) a sample comprising: (i) a microorganism (e.g., bacteria, virus, etc.) that is a source of the microbial threat, or (ii) genomic nucleic acid from a microorganism that is a source of the microbial threat; (b) determining an epigenomic sequence, epigenomic signature, or other representation thereof for the microorganism that is a source of the microbial threat; (c) comparing the epigenomic sequence, epigenomic signature, or other representation thereof to a database of microbial epigenomic sequences, epigenomic signatures, or other representations thereof, wherein the microbial epigenomic sequences, epigenomic signatures, or other representations thereof are indexed to characteristics of the respective microorganism; and (d) identifying at least one microbial characteristic of the microorganism that is a source of the microbial threat based on similarities or identities between: (i) the epigenomic sequence, epigenomic signature, or other representation thereof for the microorganism that is a source of the microbial threat, and (ii) one or more microbial epigenomic sequences, epigenomic signatures, or other representations thereof of the database; and (e) responding to the microbial threat. In some embodiments, the at least one microbial characteristic is selected from species, strain, sub-strain, serotype, virulence level, pathogenicity, origin, known geographical range, antibiotic resistance or sensitivity, and culture conditions. In some embodiments, the microbial threat is a microbial infection of an individual subject, a microbial infection or an outbreak of microbial infections across a population, or actual or potential bioterrorism. In some embodiments, responding to the microbial threat comprises treating an individual subject with an appropriate treatment, treating the infected subjects with appropriate treatments, quarantining infected subject(s), based upon one or more of the at least one microbial characteristics. In some embodiments, responding to the bacterial threat comprises alerting public health officials of the identification of subject infected with a microorganism having one or more of the at least one microbial characteristics, alerting public health officials of the identification of a population infected with microorganism having one or more of the at least one microbial characteristics, or reporting to public health officials, government officials, police, or military the identification of a microbial threat having one or more of the at least one microbial characteristics.

In some embodiments, provided herein are computer readable media or computer memory components comprising a database, wherein said database comprise at least two epigenomic sequences or signatures, wherein the at least two microbial epigenomic sequences or signatures are each correlated or indexed to one or more microbial characteristics. In some embodiments, the one or more microbial characteristics are selected from species, strain, sub-strain, serotype, virulence level, pathogenicity, origin, known geographical range, antibiotic resistance or sensitivity, and culture conditions. In some embodiments, each microbial characteristic is correlated or indexed to a sub-genomic sequence or signature within microbial epigenomic sequences or signature. In some embodiments, a processor configured to query, build, organize, etc. the database is further provided.

In some embodiments, methods of characterizing a bacteria in a sample are provided, comprising querying a database on a computer readable medium or computer memory component with a microbial epigenomic sequence or signature of the microorganism, wherein a match between the microbial epigenomic sequence or signature of the microorganism and a microbial epigenomic sequence or signature in the database identifies one or more microbial characteristics of the microorganism in the sample. In some embodiments, methods comprise querying the database of with a microbial epigenomic sequence or signature of the microorganism, wherein a match between a portion of the bacterial epigenomic sequence or signature of the microorganism and a sub-genomic microbial epigenetic sequence or signature in the database identifies one or more microbial characteristics of the microorganism in the sample.

In some embodiments, provided herein are systems comprising: (a)a sequencing module configured to perform massively-parallel, single-molecule sequencing reactions capable of detecting the epigenetic sequence of multiple nucleic acid molecules; and (b) a database comprising microbial epigenomic sequences or signatures for a plurality of microorganism, wherein each of the microbial epigenomic sequences or signatures are correlated or indexed to one or more microbial characteristics. In some embodiments, the sequencing module and the database are located at the same physical location. In some embodiments, the sequencing module and the database are located at the same physical location, but are electronically connected such that data may be sent and received between the sequencing module and the database.

In some embodiments, any of the systems and methods set forth above find use with any suitable microorganism, including but not limited to bacteria and viruses. Embodiments described herein as directed to a particular microorganism or group of microorganisms (e.g., bacteria, viruses, etc.) may find use with other microorganisms not specifically addressed in such embodiments. In some embodiments, databases, signatures, sequences, etc. that are described herein for a particular microbial group (e.g., bacteria), may also find use when applied to other microbial groups (e.g., viruses).

### Definitions

As used herein, the terms “microorganism” and “microbe” refer synonymously to any microscopic bacteria, virus, fungi, parasite, mycobacterium and/or the like.

As used herein, the term “genetic sequence” refers to a sequential listing of base identities (i.e., adenosine (A), thymine (T), guanine (G) and cytosine (C)) for all (“complete genetic sequence”) or part (“partial genetic sequence”) of a nucleic acid (e.g., DNA, RNA).

As used herein, the term “genome” refers to the complete genetic material of a species, strain, sub-strain, or organism, and includes genes as well as non-coding regions.

As used herein, the term “genomic sequence,” refers a listing (e.g., sequential) of the base identities (i.e., adenosine (A), thymine (T), guanine (G) and cytosine (C)) for the genome of a species, strain, sub-strain, or organism.

As used herein, the term “genome sequencing” refers to a single process that determines a complete genomic sequence or substantially complete genomic sequence (e.g., >90%, >91%, >93%, >94%, >95%, >96%, >97%, >98%, >99%) for a species, strain, sub-strain, or organism.

As used herein, the term “epigenetic sequence” refers to a sequential listing of base identities (i.e., adenosine (A), thymine (T), guanine (G) and cytosine (C)) as well as the position and identity of the methylated positions (e.g., 6-methyladenosine (6-mA), 4-methylcytosine (4-mC), and 5-methylcytosine (5-mC), etc.), phosphorothioated positions (e.g., sulfur replacing the non-bridging oxygen; Wang et al. PNAS (2011) vol. 108, pp. 2963-2968, herein incorporated by reference in its entirety), or other modified bases, for all or part (“partial epigenetic sequence”) of a nucleic acid (e.g., DNA, RNA).

As used herein, the terms “epigenome” and “epigenomic signature” refer to the position and identity of the methylated positions (e.g., 6-methyladenosine (6-mA), 4-methylcytosine (4-mC), and 5-methylcytosine (5-mC), etc.), phosphorothioated positions, and/or other modified positions within the genome of a species, strain, sub-strain, or organism.

As used herein, the term “epigenomic sequence” refers a listing (e.g., sequential) of the base identities (i.e., adenosine (A), thymine (T), guanine (G) and cytosine (C)) as well as the position and identity (e.g., 6-methyladenosine (6-mA), 4-methylcytosine (4-mC), and 5-methylcytosine (5-mC), etc.) of the methylated positions within the genome of a species, strain, sub-strain, or organism.

As used herein, the term “epigenomic sequencing” refers to a single process that determines a complete epigenomic sequence or substantially complete epigenomic sequence (e.g., >90%, >91%, >93%, >94%, >95%, >96%, >97%, >98%, >99%) for a species, strain, sub-strain, or organism.

As used herein, the term “partial nucleotide sequencing” refers to the determination of the positions of a subset of the bases for all or part of a nucleic acid target sequence. For example, “partial nucleotide sequencing” may comprise determining the position of the adenosines (A), thymines (T), guanines (G), cytosines (C), 6-methyladenosines (6-mA), 4-methylcytosines (4-mC), 5-methylcytosines (5-mC), or a combination thereof (e.g., methyl modified bases only) within a target nucleic acid or subsequence thereof. In some embodiments, sequencing steps performed in embodiments described herein are “partial nucleotide sequencing” steps.

As used herein, the term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, the terms “amplification free sequencing” and “non-amplification sequencing” refer to techniques for determining the genetic sequence or epigenetic sequence of a nucleic acid target without amplifying the nucleic acid target during or prior to sequencing. A variety of next generation sequencing techniques are available that do not require amplification. Typically, these techniques can also be considered “single molecule sequencing” techniques, because a sequencing read is obtained from a single molecule of target nucleic acid.

As used herein, the term “sample” refers to anything capable of being analyzed by the methods provided herein that is suspected of containing a target nucleic acid sequence. Samples may be complex samples or mixed samples, which contain nucleic acids comprising multiple different nucleic acid sequences. Samples may comprise nucleic acids from more than one source (e.g. difference species, different subspecies, etc.), subject, and/or individual. In some embodiments, the methods provided herein comprise purifying the sample or purifying the nucleic acid(s) from the sample. In some embodiments, the sample contains purified nucleic acid. In some embodiments, a sample is derived from a biological, clinical, environmental, research, forensic, or other source.

### DETAILED DESCRIPTION

Provided herein are systems and methods for determining the epigenetic sequences and signatures of microorganisms, methods of characterizing microorganisms based thereon, and methods of use thereof. Although some embodiments described herein address compositions, methods, systems, etc. for use with bacteria, such embodiments may also be applied to other suitable microorganisms (e.g., viruses).

Provided herein are compositions and methods for determining an epigenomic DNA signature of bacteria, for example, to determine bioforensic signatures for attribution, determination of virulence, development of therapeutics/diagnostics, etc. Critical information about bacteria (e.g., those involved in a bio-threat outbreak) is contained at the epigenomic level, including bacterial growth state, optimal media for cultivation, virulence, source, levels of epigenetic engineering events, etc.

Provided herein are methods and systems for characterization (e.g., attribution, virulence determination, growth conditions, etc.) of bacterial agents by obtaining an epigenetic signature (e.g., full epigenomic signature or partial epigenomic signature (e.g., random portion, targeted portion)) or epigenetic sequence (e.g., full epigenome or partial epigenome (e.g., random portion, targeted portion)) of the bacterial agent.

The methods and systems described herein provide for the determination of epigenetic data from a bacterial sample, a population of bacteria, a bacterial nucleic acid, etc., and ascribing certain features to the sample based thereon. In some embodiments, methods comprise and/or systems perform one or more steps, such as: a sample acquisition/extraction step, a bacterial culture step, a nucleic acid isolation/purification step, a nucleic acid amplification step, a sequencing (e.g., epigenomic sequencing) step, sequence organization step (e.g., identifying epigenetic signatures), comparison step, database step, characterization step (e.g., assigning features to the sample), a reporting step, etc.

In some embodiments, the methods, compositions, systems, and devices of described herein utilize samples which include, or are suspected of including, a nucleic acid sequence (e.g., bacterial sequence, unknown sequence, target sequence, etc.). Samples may be derived from any suitable source, and for purposes related to any field, including but not limited to diagnostics, research, forensics, epidemiology, pathology, archaeology, etc. A sample may be biological, environmental, forensic, veterinary, clinical, etc. in origin. A sample may be raw biological or environmental material, treated material, a bacterial culture, partially or fully-purified of isolated nucleic acid, amplified nucleic acid, etc. In some embodiments, a sample is a fixed sample (e.g., chemically fixed, paraffin embedded, etc.). In preferred embodiments, samples include one of more bacteria or nucleic acid derived from bacteria (e.g., infectious bacteria). Samples may contain, e.g., whole organisms, organs, tissues, cells (e.g., bacterial), organelles (e.g., chloroplasts, mitochondria), cell lysate, etc. A sample may contain multiple different nucleic acid sequences (e g unknown nucleic acid, target nucleic acid, template nucleic acid, non-target nucleic acid, contaminant nucleic acid, etc.) from one or more sources. Biological specimens may, for example, include whole blood, lymphatic fluid, serum, plasma, sweat, tear, saliva, sputum, cerebrospinal (CSF) fluids, amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs or washes (e.g., oral, nasopharangeal, optic, rectal, intestinal, vaginal, epidermal, etc.) and/or other biological specimens. Environmental sample may include, surface swipes, water samples, air samples, soil samples, etc.

In some embodiments, samples are mixed samples (e.g. containing nucleic acid from two or more organisms or bacterial populations). In some embodiments, samples analyzed by methods herein contain, or may contain, a plurality of different nucleic acid sequences (e.g., genetic sequences and/or epigenetic sequences). In some embodiments, a sample (e.g. mixed sample) contains one or more nucleic acid molecules (e.g. 1 . . . 10 . . . 10² . . . 10³ . . . 10⁴ . . . 10⁵ . . . 10⁶ . . . 10⁷, etc.) that contain a target sequence or an unknown sequence of interest in a particular application. In some embodiments, a sample contains zero nucleic acid molecules that contain a target sequence or an unknown sequence of interest in a particular application. In some embodiments, a sample contains nucleic acid molecules with a plurality of different sequences (e.g., genetic sequences and/or epigenetic sequences) that all contain a target sequence or unknown sequence of interest. In some embodiments, a sample contains one or more nucleic acid molecules (e.g. 1 . . . 10 . . . 10² . . . 10³ . . . 10⁴ . . . 10⁵ . . . 10⁶ . . . 10⁷, etc.) that do not contain a target sequence or unknown sequence of interest in a particular application.

In some embodiments, bacteria are isolated and/or purified from a sample. In some embodiments, isolated bacteria are analyzed without culturing or expanding the isolated population. In some embodiments, bacteria from a sample are cultured prior to epigenetic analysis. In some embodiments, culture conditions are selected based on the type of bacteria and/or the desired analysis. In some embodiments, bacteria are cultured under multiple different sets of conditions (e.g., stress conditions, rich conditions, supplemented conditions (e.g., serum supplemented), etc.) and the epigenetic signatures of the bacteria under the different conditions are compared.

The systems and methods described herein find use in the analysis and characterization of any suitable bacteria of sample. For example, a sample may comprise a one or more types of bacteria selected from the list including, but not limited to: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia group, Aeromonas hydrophilia, Escherichia coli, Citrobacte freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Acinetobacter anitratisYersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcusagalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus. In certain embodiments, a sample does not contain bacteria, but instead comprises bacterial nucleic acid (e.g., complete genomic bacterial nucleic acid), for example, from one of the aforementioned species of bacteria.

In some embodiments, nucleic acid is extracted, isolated, and/or purified from a sample prior to epigenetic analysis. Various bacterial DNA extraction techniques are well known to those skilled in the art. In some embodiments, methods and systems provide nucleic acid analysis (e.g., epigenetic sequencing) from raw sample (e.g., biological fluid, sample with environmental contaminant, whole bacteria, bacterial lysate, etc.) without processing or with limited processing.

In some embodiments, all or a portion of the nucleic acid from a sample is directly sequenced (e.g., epigenetic sequencing), without one or more of amplification and/or reverse transcription. Since epigenetic alterations (e.g., methylation, phophorothioation, etc.) of the DNA are typically lost via amplification, nucleic acid analysis techniques that maintain and detect the epigenetic signature of the nucleic acid are utilized.

In other embodiments, all or a portion of the nucleic acid from a sample is amplified and/or reverse transcribed prior to or following analysis (e.g., for genetic sequencing (e.g., non-epigenetic sequencing), for comparison to non-amplified nucleic acid, for other analysis, etc.). Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA). Amplifications used in method or assays described herein may be performed in bulk and/or partitioned volumes (e.g. droplets). Further, amplification reactions may be performed using thermal cycling (e.g., PCR, RT-PCR, LCR, etc.) and/or isothermally (e.g., branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, etc.).

The polymerase chain reaction, commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. Other amplification/transcription techniques that may find use in embodiments described herein, either alone or in combination, are addressed below.

Transcription mediated amplification, commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. In a variation, TMA optionally incorporates the use of blocking moieties, terminating moieties, and other modifying moieties to improve TMA process sensitivity and accuracy.

The ligase chain reaction, commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification, commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method.

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβreplicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety).

In some embodiments, provided herein are systems and methods useful in detection of both the nucleotide sequence (e.g., A, C, G, T) and epigenetic modifications (e.g., 6-mA, 4-mC, 5-mC, phosphorothioation, etc.) of nucleic acid sample (e.g., from a bacteria). For example, nucleotides within sequence templates are detected during nucleic acid sequencing reactions through the use of single molecule nucleic acid analysis such that the resulting sequence read(s) comprising both genetic and epigenetic sequence data. The epigenetic data is indicative of not only the position of a modification (e.g., methylated base, phophorothioation, etc.), but also the type of base modification. In some embodiments, epigenetic data is obtained using techniques (e.g., single molecule sequencing techniques), without the need for comparison to a non-modified sequence, e.g., as in conventional bisulfite sequencing. In other embodiments, a technique that utilizes modification of the methylated nucleotides is used to obtain epigenetic data (e.g., bisulfite modification is described in U.S. Pat. No. 6,017,704, the entire disclosure of which is incorporated herein by reference). In some embodiments, a single read from a single molecule, a plurality of reads from a single molecule, or a single read from multiple single molecules is sufficient to provide both the genetic and epigenetic data from a nucleic acid and/or bacterial sample. In some embodiments, the epigenetic data is collected over the entire bacterial genome (e.g., epigenomic data).

Nucleic acid molecules may be analyzed by any number of techniques to determine the genetic and/or epigenetic sequence. The analysis may identify the sequence (e.g., genetic or epigenetic) of all or a part of a nucleic acid. In some embodiments, analysis determines the genomic and/or epigenomic sequence for a sample organism or a species, strain, or sub-strain in general. Any techniques capable of determining genetic sequence and/or modification (e.g., methylation, phophorothioation, etc.) status of a nucleic acid may find use in embodiments herein. To the extent that sequencing technique not capable of determining epigenetic status of a nucleic acid are described herein, application of these techniques in embodiments described herein is limited to application in which only genetic data and not epigenetic data is to be obtained.

Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack, experimentally RNA is usually, although not necessarily, reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties) the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties) and the Adessi PCR colony technology (Adessi et al. (2000). Nucleic Acid Res. 28, E87; WO 00018957; herein incorporated by reference in its entirety).

In some embodiments, chain terminator sequencing is utilized. Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A set of methods referred to as “next-generation sequencing” techniques have emerged as alternatives to Sanger and dye-terminator sequencing methods (Voelkerding et al., Clinical Chem., 55: 641-658, 2009; MacLean et al., Nature Rev. Microbiol., 7: 287-296; each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, Pacific Biosciences (PAC BIO RS II) and other platforms commercialized by VisiGen, Oxford Nanopore Technologies Ltd., and, respectively. In some embodiments, due to the requirement that the epigenetic signature of the nucleic acid be maintained and determined, sequencing techniques that do not require or utilize amplification of the nucleic acid are particularly preferred.

One real-time single molecule sequencing system developed by Pacific Biosciences (Voelkerding et al., Clinical Chem., 55: 641-658, 2009; MacLean et al., Nature Rev. Microbiol., 7: 287-296; U.S. Pat. No. 7,170,050; U.S. Pat. No. 7,302,146; U.S. Pat. No. 7,313,308; U.S. Pat. No. 7,476,503; all of which are herein incorporated by reference) utilizes reaction wells 50-100 nm in diameter and encompassing a reaction volume of approximately 20 zeptoliters (10×10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera. In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10-21 liters). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background. Variations on the real-time single molecule sequencing system developed by Pacific Biosciences (SMRT, ZMWs, etc.), and combinations with other systems and methods are also within the scope of embodiments described herein.

In another next-generation sequencing technique, pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotiter plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 1×10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color and thus identity of each probe corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing in employed (see, e.g., Astier et al., J Am Chem Soc. 2006 Feb. 8; 128(5):1705-10, herein incorporated by reference). The theory behind nanopore sequencing has to do with what occurs when the nanopore is immersed in a conducting fluid and a potential (voltage) is applied across it: under these conditions a slight electric current due to conduction of ions through the nanopore can be observed, and the amount of current is exceedingly sensitive to the size of the nanopore. If DNA molecules pass (or part of the DNA molecule passes) through the nanopore, this can create a change in the magnitude of the current through the nanopore, thereby allowing the sequences of the DNA molecule to be determined.

Another exemplary nucleic acid sequencing approach that may be adapted for use with the systems, devices, and methods was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Patent Publication No. 20090035777, entitled “HIGH THROUGHPUT NUCLEIC ACID SEQUENCING BY EXPANSION,” that was filed Jun. 19, 2008, which is incorporated herein in its entirety.

Other emerging single molecule sequencing methods include real-time sequencing by synthesis using a VisiGen platform (Voelkerding et al., Clinical Chem., 55: 641-658, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Processes and systems for such real time sequencing that may be adapted for use with the invention are described in, for example, U.S. Pat. Nos. 7,405,281, entitled “Fluorescent nucleotide analogs and uses therefor”, issued Jul. 29, 2008 to Xu et al., 7,315,019, entitled “Arrays of optical confinements and uses thereof”, issued Jan. 1, 2008 to Turner et al., U.S. Pat. No. 7,313,308, entitled “Optical analysis of molecules”, issued Dec. 25, 2007 to Turner et al., U.S. Pat. No. 7,302,146, entitled “Apparatus and method for analysis of molecules”, issued Nov. 27, 2007 to Turner et al., and U.S. Pat. No. 7,170,050, entitled “Apparatus and methods for optical analysis of molecules”, issued Jan. 30, 2007 to Turner et al., U.S. Patent Publications Nos. 20080212960, entitled “Methods and systems for simultaneous real-time monitoring of optical signals from multiple sources”, filed Oct. 26, 2007 by Lundquist et al., 20080206764, entitled “Flowcell system for single molecule detection”, filed Oct. 26, 2007 by Williams et al., 20080199932, entitled “Active surface coupled polymerases”, filed Oct. 26,2007 by Hanzel et al., 20080199874, entitled “CONTROLLABLE STRAND SCISSION OF MINI CIRCLE DNA”, filed Feb. 11,2008 by Otto et al., 20080176769, entitled “Articles having localized molecules disposed thereon and methods of producing same”, filed Oct. 26, 2007 by Rank et al., 20080176316, entitled “Mitigation of photodamage in analytical reactions”, filed Oct. 31, 2007 by Eid et al., 20080176241, entitled “Mitigation of photodamage in analytical reactions”, filed Oct. 31, 2007 by Eid et al., 20080165346, entitled “Methods and systems for simultaneous real-time monitoring of optical signals from multiple sources”, filed Oct. 26, 2007 by Lundquist et al., 20080160531, entitled “Uniform surfaces for hybrid material substrates and methods for making and using same”, filed Oct. 31, 2007 by Korlach, 20080157005, entitled “Methods and systems for simultaneous real-time monitoring of optical signals from multiple sources”, filed Oct. 26, 2007 by Lundquist et al., 20080153100, entitled “Articles having localized molecules disposed thereon and methods of producing same”, filed Oct. 31, 2007 by Rank et al., 20080153095, entitled “CHARGE SWITCH NUCLEOTIDES”, filed Oct. 26, 2007 by Williams et al., 20080152281, entitled “Substrates, systems and methods for analyzing materials”, filed Oct. 31, 2007 by Lundquist et al., 20080152280, entitled “Substrates, systems and methods for analyzing materials”, filed Oct. 31, 2007 by Lundquist et al., 20080145278, entitled “Uniform surfaces for hybrid material substrates and methods for making and using same”, filed Oct. 31, 2007 by Korlach, 20080128627, entitled “SUBSTRATES, SYSTEMS AND METHODS FOR ANALYZING MATERIALS”, filed Aug. 31, 2007 by Lundquist et al., 20080108082, entitled “Polymerase enzymes and reagents for enhanced nucleic acid sequencing”, filed Oct. 22, 2007 by Rank et al., 20080095488, entitled “SUBSTRATES FOR PERFORMING ANALYTICAL REACTIONS”, filed Jun. 11, 2007 by Foquet et al., 20080080059, entitled “MODULAR OPTICAL COMPONENTS AND SYSTEMS INCORPORATING SAME”, filed Sep. 27, 2007 by Dixon et al., 20080050747, entitled “Articles having localized molecules disposed thereon and methods of producing and using same”, filed Aug. 14, 2007 by Korlach et al., 20080032301, entitled “Articles having localized molecules disposed thereon and methods of producing same”, filed Mar. 29, 2007 by Rank et al., 20080030628, entitled “Methods and systems for simultaneous real-time monitoring of optical signals from multiple sources”, filed Feb. 9, 2007 by Lundquist et al., 20080009007, entitled “CONTROLLED INITIATION OF PRIMER EXTENSION”, filed Jun. 15, 2007 by Lyle et al., 20070238679, entitled “Articles having localized molecules disposed thereon and methods of producing same”, filed Mar. 30, 2006 by Rank et al., 20070231804, entitled “Methods, systems and compositions for monitoring enzyme activity and applications thereof”, filed Mar. 31, 2006 by Korlach et al., 20070206187, entitled “Methods and systems for simultaneous real-time monitoring of optical signals from multiple sources”, filed Feb. 9, 2007 by Lundquist et al., 20070196846, entitled “Polymerases for nucleotide analogue incorporation”, filed Dec. 21, 2006 by Hanzel et al., 20070188750, entitled “Methods and systems for simultaneous real-time monitoring of optical signals from multiple sources”, filed Jul. 7, 2006 by Lundquist et al., 20070161017, entitled “MITIGATION OF PHOTODAMAGE IN ANALYTICAL REACTIONS”, filed Dec. 1, 2006 by Eid et al., 20070141598, entitled “Nucleotide Compositions and Uses Thereof”, filed Nov. 3, 2006 by Turner et al., 20070134128, entitled “Uniform surfaces for hybrid material substrate and methods for making and using same”, filed Nov. 27, 2006 by Korlach, 20070128133, entitled “Mitigation of photodamage in analytical reactions”, filed Dec. 2, 2005 by Eid et al., 20070077564, entitled “Reactive surfaces, substrates and methods of producing same”, filed Sep. 30, 2005 by Roitman et al., 20070072196, entitled “Fluorescent nucleotide analogs and uses therefore”, filed Sep. 29, 2005 by Xu et al., and 20070036511, entitled “Methods and systems for monitoring multiple optical signals from a single source”, filed Aug. 11, 2005 by Lundquist et al., and Korlach et al. (2008) “Selective aluminum passivation for targeted immobilization of single DNA polymerase molecules in zero-mode waveguide nanostructures” Proc. Nat'l. Acad. Sci. U.S.A. 105(4): 11761181—all of which are herein incorporated by reference in their entireties.

In some embodiments, nucleic acids are analyzed by determination of their mass and/or base composition. For example, in some embodiments, nucleic acids are detected and characterized by the identification of a unique base composition signature (BCS) using mass spectrometry (e.g., Abbott PLEX-ID system, Abbot Ibis Biosciences, Abbott Park, Ill.,) described in U.S. Pat. Nos. 7,108,974, 8,017,743, and 8,017,322; each of which is herein incorporated by reference in its entirety. In some embodiments, a MassARRAY system (Sequenom, San Diego, Calif.) is used to detect or analyze sequences (See e.g., U.S. Pat. Nos. 6,043,031; 5,777,324; and 5,605,798; each of which is herein incorporated by reference).

In certain embodiments, the Ion Torrent sequencing technology is employed. The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a fragment of the NGS fragment library to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

In some embodiments, a sample comprising bacterial DNA is treated to fragment the DNA, and the resulting fragments (e.g., in a single reaction mixture) are sequenced (e.g., single-molecule, real-time sequencing) to yield both genetic and epigenetic sequences of the fragments. In some embodiments, a single sequencing read corresponds to a single fragment molecule. In some embodiments, a sequencing read is obtained for each fragment molecule (e.g., bacterial genomic fragment) sequenced. In some embodiments, epigenetic signatures are generated from the fragment sequences. In some embodiments, a genomic and/or epigenomic sequences are reconstructed based upon a plurality of fragment data (e.g., overlapping fragments). In some embodiments, a genomic and/or epigenomic signature is reconstructed based upon a plurality of fragment data (e.g., overlapping fragments).

Raw data obtained from sequencing is converted into epigenetic data (e.g., epigenetic sequence, epigenomic sequence, epigenetic signature, epigenomic signature, etc.). In some embodiments, the epigenetic data from a sample (e.g., bacteria, bacterial population, nucleic acid, etc.) is queried to identify markers indicative of various features of the source bacteria (e.g., attribution, virulence, antibiotic resistance/sensitivity, growth conditions, etc.). In some embodiments, the epigenetic data is searched for the presence of particular markers (e.g., sequences, methylation sites, combinations thereof, etc.) that correspond to features of interest. In other embodiments, epigenetic data obtained from a sample is queried against control epigenetic data from bacteria with known features.

In some embodiments, epigenetic data obtained from a sample (e.g., containing nucleic acid from multiple bacteria types, containing unknown number and/or types of bacteria, etc.) is queried for the presence of a particular type of bacteria (e.g., a virulent strain involved in an outbreak, an antibiotic resistant strain, a strain not yet observed in a particular region, etc.).

In certain embodiments, epigenetic data obtained from a sample (e.g., an epigenetic sequence, epigenomic signature, or epigenomic sequence) is compared to a database for characterization of one or more features. Suitable databases for use in characterization of bacterial agents via epigenetics include databases of bacterial particular epigenomic signatures; databases of complete, substantially complete (e.g., >90% to >99%) or partial epigenomic sequences; databases of complete, substantially complete (e.g., >90% to >99%) or partial epigenomic signatures; databases of potentially methylated positions; etc. Databases may correlate such epigenetic information with one or more characterizing features, including but not limited to: identification (e.g., species, strain, sub-strain, etc.), degree of virulence, type/degree of antibiotic resistance/sensitivity, growth state, optimal growth conditions, origin, level of epigenetic engineering, locations/regions/nations exposed, etc. In some embodiments, determining epigenetic sequence or signature information and querying such a database allow bioforensic characterization of a bacterial agent.

In some embodiments, rather than using full sequences (e.g., epigenomic sequences), other representations (e.g., epigenetic or epigenomic signatures) are used (e.g., for querying, for storing in a database, etc.). In some embodiments, epigenetic signatures retain the epigenetic information of a sequence, but with less genetic data (e.g., non-modified positions are not present). In some embodiments, signatures require less storage space and less computing power to work with. In some embodiments, epigenetic sequences are converted to epigenetic signatures. In some embodiments, both epigenetic sequences and epigenetic signatures are utilized for particular steps in methods described herein. An epigenetic signature may comprise only the position and identity of modified (e.g., methylated, phophorothioation, etc.) nucleotides in a nucleic acid sequence. In some embodiments, and epigenetic signature comprises the position and identity of modified (e.g., methylated, phophorothioated, etc.) nucleotides and those that are not modified in the particular variant nucleic acid sequence but are in other variants. In some embodiments, an epigenetic signature may comprise another useful representation of data contained in the epigenetic sequence. In some embodiments, an epigenomic signature is a representation of the epigenetic data contained within the genome.

In some embodiments, a database contains full epigenomic sequences or full epigenomic signatures for a group of bacterial agents (e.g., the strains of a single species, multiple related species, etc.). A match of a queried sequence with an entry in the database provides a user (e.g., researcher, clinician, etc.) with all features correlated with the queried epigenetic information. In other embodiments, a database contains specific epigenetic positions and/or signature segments that correlate with features of interest (e.g., degree of virulence, specific drug resistances, etc.). In other embodiments, a database is specific to a particular feature(s), and epigenetic data is queried against the database to characterize a sample with regard to that specific feature (e.g., virulence, resistance/sensitivity, growth conditions, etc.).

In some embodiments, a perfect match between an epigenetic signature, epigenetic sequence, or epigenetic sequence in a sample correlates the bacteria (e.g., target bacteria, unknown bacteria, etc.) with the features identified in the database as corresponding to such signature or sequence. In some embodiments, a partial match (e.g., >99%, >98%, >97%, >96%, >95%, >94%, >93%, >92%, >91%, >90%, >85%, >80%, >75%, >70%, >60%, >50%) between all or a key portion of an epigenetic signature, epigenetic sequence, or epigenetic sequence in a sample correlates the bacteria (e.g., target bacteria, unknown bacteria, etc.) with the features identified in the database as corresponding to such signature or sequence. In some embodiments, a confidence level is identified/provided for the correlation between a signature/sequence and a particular feature based on the epigenetic identity. In some embodiments, a database identifies multiple epigenetic sequences and/or signatures that correlate to a particular feature and similarity/difference to these multiple sequences allows more accurate correlation to the feature (e.g., an epigenetic sequence with >90% epigenetic identity to three sequences from different strains exhibiting a feature (e.g., resistance to a particular antibiotic) has a greater likelihood of being from a bacteria exhibiting that feature than a bacterial with nucleic acid similar to only one sequence with such a feature).

In some embodiments, epigenomic sequences or signatures are queried against a database of known genomic sequences. In such embodiments, a match between the sample sequence and one in the database allows one or more features from the database sequence (and the bacteria from which it was derived) to be ascribed to the sample bacteria. In other embodiments, epigenomic sequences or signatures are queried against a database of subgenomic epigenetic sequences or signatures, in which each subgenomic portion in the database correlates to one or more features. In such embodiments, a sample genomic sequence or signature may correlate with multiple different database entries, corresponding to different portion of the sample sequence.

In some embodiments, subgenomic epigenetic sequence or signature are queried against a database of known genomic sequences. In such embodiments, a match between the sample sequence and an epigenomic sequence or signature in the database allows one or more features from the database sequence (e.g., those correated to that region of the nucleic acid) to be ascribed to the sample bacteria. In other embodiments, subgenomic epigenetic sequences or signatures are queried against a database of subgenomic epigenetic sequences or signatures, in which each subgenomic portion in the database correlates to one or more features. In such embodiments, a subgenomic epigenetic sequence or signature is directly correlated with a database entry and the features ascribed thereto.

In some embodiments, methods are provided for developing and/or populating the epigenetic databases utilized in embodiments described herein. In some embodiments, databases are compiled from known epigenetic or epigenomic sequences, and the bacterial features known to correlate thereto. In some embodiments, effort is taken to construct a database by empirically determining epigenomic or epigenetic sequences and/or signatures and correlating such data to bacterial features. In some embodiments, such correlation is computationally automated.

In certain embodiments, upon querying a database with epigenetic information not contained therein, the query is populated into the database. In some such embodiments, features of the newly added entry are populated by comparison to the database or other databases. In some embodiments, the database is self-populating, because querying the database generates new entries into the database. In other such embodiments, features of newly added entries are manually populated.

In some embodiments, a master database comprising multiple epigenetic sequences, epigenomic sequences, epigenetic signatures, and/or epigenomic signatures correlated with characteristics and features (e.g., species, strain, sub-strain, origin, virulence, resistance, growth conditions, etc.) for each is provided. The master database may be organized (e.g., automatically based, e.g., on a query, manually by an operator, combinations thereof, etc.) into sub-databases for particular applications, uses, or queries. For example, a sub-database of a particular group of bacteria (e.g., gram negatives, Enterobacteriaceae, etc.), a species of bacteria (e.g., Salmonella bongori, Salmonella enterica, etc.), a particular features (e.g., resistance to chloramphenicol, increased virulence, prior detection in a region, etc.), a set of features (e.g., virulence and drug resistance), an epigenetic marker (e.g., 6-mA at a particular position, etc.), or a group of epigenetic markers. In some embodiments, a sub-database is produced and queried to reduce computational time.

In some embodiments, all or a portion of the methods described herein are provided as a service. In some embodiments, a user (e.g., a clinician, investigator, researcher, etc.) arranges, contracts, pays, etc. to have a sample (e.g., biological sample, environmental sample, bacterial sample, nucleic acid sample, etc.) and/or epigenetic data (e.g., sequence, signature, etc) analyzed. In some embodiments, a sample is submitted (e.g., in-person, via mail or courier, etc.) and sequencing of nucleic acid (e.g., epigenetic sequencing, determination of an epigenetic signature) is performed by the service (e.g., at a diagnostic testing facility, at a government laboratory, etc.). In some embodiments, data (e.g., epigenetic sequence, epigenetic signature, epigenomic sequence, raw data, etc.) collected by a user (e.g., a clinician, investigator, researcher, etc.) are submitted to a testing facility for analysis (e.g., identification of particular signatures (e.g., virulence profile, resistance profile, origin, etc.), comparison to a database, characterization of features, etc.). Embodiments described herein include any suitable combination of user-performed and service-performed steps. In some embodiments, methods described herein comprise of consist of only the steps performed by either the user of the service (e.g., sample collection, sample analysis, data collection, data analysis, feature identification, etc.). In some embodiments, any combination of steps may be performed by a user and/or service.

In some embodiments, based on analysis of the epigenetic data from a sample and/or comparison to a control or database, the sample and/or bacteria therein are characterized (e.g., ascribed certain functional or physical features). In some embodiments, features correlated to epigenetic data include, but are not limited to: species, strain, substrain, serotype, geographic source, pathogenicity, virulence (e.g., hypervirulence), resistance/sensitivity (e.g., multiresistance), sporulation conditions, mitotic initiation conditions (e.g., from spore), [* * * PLEASE INDICATE OTHER CHARACTERISTICS]

In some embodiments, epigenetic data correlates to a bacteria's resistance or sensitivity to an antibiotic or class of antibiotics. Examples of the antibacterial antibiotics, for which resistence/sensitivity may be identified by epigenetic analysis include, but are not limited to: aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicois (e.g., azidamfenicol, chloramphenicol, florfenicol, thiamphenicol), ansamycins (e.g., rifamide, rifampin, rifamycin sv, rifapentine, rifaximin), .beta.-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, ceifuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefiiietazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, mikamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Analysis of epigenetic data (e.g., comparison of an epigenetic signature/sequence to a database) in pathogenic organisms can identify the biological basis for their pathogenicity. This insight can be used to determine appropriate treatments, or to develop new treatments for combating individual infections and widespread outbreaks.

In some embodiments, epigenetic signatures (e.g., epigenomic signatures) are responsive to environmental factors. In some embodiments, characterization (e.g., via database analysis and query) of epigenetic signatures influenced by environmental factors find use in understanding the nature of a bacterial sample (e.g., source, attribution, etc.), and may provide a diagnostic/screening methods.

In some embodiments, epigenetic data (e.g., epigenomic sequence/signature) for a bacteria or population is analyzed for growth-condition-dependent epigenetic modifications.

For example, epigenetic data is collected from two or more bacteria samples cultured under different culture conditions (e.g., rich media, stress media, supplemented media (e.g., serum supplemented), etc.), and the epigenetic data (e.g., epigenomic sequence/signature) are compared to identify condition dependent epigenetic modifications In some embodiments, condition-dependent modifications are compared between bacterial populations, species, strains, etc. In some embodiments, a database of condition-dependent modifications from different bacterial populations allows for identification of traits for a particular bacteria queried against the database.

In some embodiments, the results of sequencing (epigenetic sequencing) and analysis are reported (e.g., to a user, clinician, researcher, investigator, etc.). Bacterial characteristic and/or epigenetic data (e.g., epigenomic signature) are identified and/or reported as an outcome/result of an analysis. An outcome or result may be produced by receiving data (e.g., epigenetic sequence data) and/or information (e.g., know about the bacterial sample), transforming the data and/or information and provide an outcome or result (e.g., by comparison to a database). An outcome or result may be determinative of an action to be taken in order to respond to a particular bacteria (e.g., infection, outbreak, bio-threat, etc.). In some embodiments, characteristics identified by methods described herein can be independently verified by further testing (e.g., phenotypic validation).

In some embodiments, analysis results are reported (e.g., to a health care professional (e.g., laboratory technician or manager; physician, nurse, or assistant, etc.), researcher, investigator, etc.). In some embodiments, a result is provided on a peripheral, device, or component of an apparatus. For example, sometimes an outcome is provided by a printer or display. In some embodiments, an outcome is reported in the form of a report, and in certain embodiments the report comprises a display of bacterial characteristics, risk assessment, action items, confidence parameters, etc. Generally, an outcome can be displayed in a suitable format that facilitates downstream use of the reported information. Non-limiting examples of formats suitable for use for reporting and/or displaying data, characteristics, etc. include text, outline, digital data, a graph, graphs, a picture, a pictograph, a chart, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, and combination of the foregoing.

Generating and reporting results from the generation and analysis of epigenetic data comprises transformation of nucleic acid sequence reads into a representation of the characteristics of a bacteria or bacterial population. Such a representation reflects information not determinable from the nucleic acid in the absence of the method steps described herein. Converting nucleic acid into feature information allows actions to be taken in response to a bacterial infection, outbreak, or threat. As such, these method and systems provided herein address the problem of rapidly identifying and understanding a bacterial threat (e.g., infection, outbreak, bioterror agent, etc.) that confronts the fields of medicine, security, public health, national defense, anti-terrorism, epidemiology, etc.

In some embodiments, a user or a downstream individual, upon receiving or reviewing a report comprising one or more results determined from the analyses provided herein, with take specific steps or actions in response. For example, a health care professional or qualified individual may test a subject or patient for infection or response to treatment. A public health official may issue a notification or take steps to prevent the spread of an outbreak. A security official may take steps to prevent the deployment or use of an agent. The present invention is not limited by the number of ways or fields in which the technology herein may find use.

The term “receiving a report” as used herein refers to obtaining, by a communication means, a written and/or graphical representation comprising results or outcomes of epigenetic analysis. The report may be generated by a computer or by human data entry, and can be communicated using electronic means (e.g., over the internet, via computer, via fax, from one network location to another location at the same or different physical sites), or by a other method of sending or receiving data (e.g., mail service, courier service and the like). In some embodiments the outcome is transmitted in a suitable medium, including, without limitation, in verbal, document, or file form. The file may be, for example, but not limited to, an auditory file, a computer readable file, a paper file, a laboratory file or a medical record file. A report may be encrypted to prevent unauthorized viewing.

As noted above, in some embodiments, systems and method described herein transform data from one form into another form (e.g., from a nucleic acid to actual features of a bacteria, from epigenetic sequence to an epigenetic signature, etc.). In some embodiments, the terms “transformed”, “transformation”, and grammatical derivations or equivalents thereof, refer to an alteration of data from a physical starting material (e.g., bacterial population, sample nucleic acid, etc.) into a digital representation of the physical starting material (e.g., sequence read data), a sequential representation of that starting material (e.g., epigenetic or epigenomic sequence), a condensation of the sequential representation (e.g., epigenetic or epigenomic signature), or a characteristic description of that starting material. In some embodiments, transformation involves conversion of data between any of the above mention representations of the physical nucleic acid.

Certain processes and methods described herein (e.g., data acquisition, epigenetic sequence/signature determination, communication, categorizing, database querying, database management, database population, feature correlation, etc.) are performed by (or cannot be performed without) a computer, processor, software, module and/or other device. Methods described herein typically are computer-implemented methods, and one or more portions of a method sometimes are performed by one or more processors. In some embodiments, an automated method is embodied in software, processors, peripherals and/or an apparatus comprising the like, that determine epigenetic sequence reads, epigenetic signature, database comparisons, feature correlation, etc.

As used herein, software refers to computer readable program instructions that, when executed by a processor, perform computer operations, as described herein.

Epigenetic sequence, epigenetic signatures, and epigenomic information are referred to herein as “data” or “data sets.” In some embodiments, data or data sets can be characterized are analyzed (e., by comparison to a database) in order to ascribe one or more features to the bacterial source of the sample nucleic acid.

Apparatuses, software and interfaces may be used to conduct methods described herein. In some embodiments, such hardware and software components allow automation of one or more steps of the methods described herein. Using apparatuses, software and interfaces, a user may, for example, process a raw sample (e.g., remove contaminants), purify/isolate nucleic acid, collect data from a nucleic acid, convert direct-read data to a sequence or signature, determine an epigenetic sequence or signature, send data (e.g., between computers, facilities, users, services, etc.), query a database, populate a database, ascribe features, report results, make recommendations, etc.

A system typically comprises one or more devices or apparatus. Each device/apparatus often comprises components selected from memory, processor(s), display, user interface, etc. Where a system includes two or more devices/apparatuses, some or all of the various components of the system may be located at different locations. Where a system includes two or more devices/apparatuses, some or all of the apparatus may be located at the same location as a user, some or all of the apparatus may be located at a location different than a user, all of the apparatus may be located at the same location as the user, and/or all of the apparatus may be located at one or more locations different than the user.

A system sometimes comprises one or more computing apparatuses (e.g., data analysis apparatus, database-containing apparatus, etc.) and a sequencing apparatus, where the sequencing apparatus is configured to receive physical nucleic acid and generate epigenetic sequence reads, and the computing apparatus is configured to process/analyze the epigenetic information obtained from the sequencing apparatus. A computing apparatus sometimes is configured to compare epigenetic data from a sample to a database and to ascribe various features based thereon.

A user may, for example, place a query to software which then may acquire a data set (e.g., a database, a control sequence, an epigenetic data set from a bacterial sample, etc.) via internet access, and in certain embodiments, a programmable processor may be prompted to acquire a suitable data set based on given parameters (e.g., epigenetic signatures for bacteria having a particular feature or set of features. A programmable processor also may prompt a user to select one or more data set options or database options selected by the processor based on given parameters. A programmable processor may prompt a user to select one or more data set options or database options selected by the processor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, apparatuses, or computer programs.

Systems described herein may comprise general components of computer systems, such as, for example, network servers, laptop systems, desktop systems, handheld systems, personal digital assistants, tablets, smart phones, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input (e.g., from a user, from a sequencer, from a database, etc.) and output means may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user (e.g., clinician, researcher, investigator, etc.) accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system includes a communications interface in certain embodiments. A communications interface allows for transfer of software and data (e.g., epigenetic data, database information, query results, identified bacterial features, etc.) between a computer system and one or more external devices. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels, wireless. As an example, a communications interface may be used to receive signal information that can be detected by a signal detection module.

In some embodiments, output from a sequencing apparatus may serve as data that can be input via an input device. In certain embodiments, epigenetic sequence is data that is input input via an input device. In certain embodiments, nucleic acid fragment size (e.g., length) is data that is input via an input device. In certain embodiments, simulated data is generated by an in silico process and the simulated data is input via an input device. The term “in silico” refers to research and experiments performed using a computer. In silico processes include, but are not limited to, simulated epigenetic sequences (e.g., generated from a database of known sequences based on particular desired features).

A system may include software useful for performing a process described herein, and software may include one or more modules for performing such processes (e.g., sequencing module, query module, data display module, user (e.g., clinician, researcher, investigator) interface module). The term “software” refers to computer readable program instructions that, when executed by a computer, perform computer operations. Instructions executable by the one or more processors sometimes are provided as executable code, that when executed, can cause one or more processors to implement a method described herein. A module described herein can exist as software, and instructions (e.g., processes, routines, subroutines) embodied in the software can be implemented or performed by a processor. For example, a module (e.g., a software module) can be a part of a program that performs a particular process or task. The term “module” refers to a self-contained functional unit that can be used in a larger apparatus or software system. A module can comprise a set of instructions for carrying out a function of the module. A module can transform data and/or information. Data and/or information can be in a suitable form. A module can accept or receive data and/or information, transform the data and/or information into a second form, and/or provide or transfer the second form to an apparatus, peripheral, component or another module. A module can perform one or more of the following non-limiting functions, for example: obtaining epigenetic sequence data (e.g., from a sample), generating an epigenetic signature (e.g., from sequence data), generating epigenomic data (e.g., from multiple sub-genomic nucleic sequences), assembling genomic sections, normalizing (e.g., normalizing reads), comparing two or more epigenetic data sets, populating a database, creating a sub-database from a master database (e.g., based on desired sequence, signature, features, species, strain, substrain, etc.), querying a database, identification, attribution, characterization (e.g., virulence level, resistance/sensitivity, origin, etc.), categorizing, plotting, determining an outcome, recommending a plan of action, etc. A processor can, in some instances, carry out the instructions in a module. In some embodiments, one or more processors are required to carry out instructions in a module or group of modules. A module can provide data and/or information to another module, apparatus or source and can receive data and/or information from another module, apparatus or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory).

An apparatus, in some embodiments, comprises at least one processor for carrying out the instructions in a module. In some embodiments, epigenetic data (e.g., a database of epigenetic data correlated to bacterial features) are accessed by a processor that executes instructions configured to carry out a method described herein. In some embodiments, epigenetic data accessed by a processor is stored within memory of a system, and the data is accessed locally or remotely for query (e.g., with sample epigenetic data), manipulation, analysis, organization (e.g., formation of sub-databases). In some embodiments, an apparatus comprising a module receives and/or transfers epigenetic data and/or analysis thereof to and from other modules. In some embodiments, an apparatus comprises peripherals and/or components. In some embodiments, an apparatus can comprise one or more peripherals or components that can transfer data and/or information to and from other modules, peripherals and/or components. In some embodiments, an apparatus interacts with a peripheral and/or component that provides data and/or information. In some embodiments, peripherals and components assist an apparatus in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a processor, a server, CDs, DVDs, graphic cards, specialized I/O devices (e.g., sequencers, photo cells, photo multiplier tubes, optical readers, sensors, etc.), one or more flow cells, fluid handling components, sequencer, network interface controllers, ROM, RAM, wireless transfer methods and devices (Bluetooth, WiFi, and the like), the world wide web (www), the internet, a computer and/or another module.

In some embodiments, systems described herein comprise one or more of a sequencing module, an analysis module, a processing module, and data display module, which are utilized in carrying out the methods described herein. Other non-limiting examples of system modules include: logic processing module, data organization module, amplification module, sample handling module, sample purification module, normalization module, comparison module, memory module, database module, categorization module, adjustment module, plotting module, outcome module, and submodules or combination thereof. In some embodiments, data is transferred between modules and analyzed therein to carry our methods described herein.

The terms “obtaining,” “transferring,” “receiving,” etc. refer to movement of data (e.g., raw sequence data, epigenetic sequence, epigenetic signature, bacterial features, query requests, etc.) between modules, devices, apparatuses, etc. within a system. These terms may also refer to the handling of samples and purified versions thereof (e.g., with respect to amplification, purification, and/or sequencing modules). Input information may be generated in the same location at which it is received, or it may be generated in a different location and transmitted to the receiving location. In some embodiments, input information is modified before it is processed (e.g., placed into a format amenable to processing (e.g., tabulated)). In some embodiments, provided are computer program products, such as, for example, a computer program product comprising a computer usable medium having a computer readable program code embodied therein, the computer readable program code adapted to be executed to implement a method comprising, for example, the general steps of: (a) obtaining epigenetic sequence data from a nucleic acid from a bacterial sample; (b) generating an epigenomic sequence or signature from the epigenetic data; (c) comparing the epigenomic sequence or signature to a control or database; (d) characterizing said bacterial sample.

Software may include one or more algorithms in certain embodiments. An algorithm may be used for processing epigenetic sample data and stored data, analyzing data, and/or providing an outcome or report according to a sequence of instructions. An algorithm often is a list of defined instructions for completing a task. Starting from an initial state, the instructions may describe a computation that proceeds through a defined series of successive states, eventually terminating in a final ending state. By way of example, and without limitation, an algorithm may be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. In some embodiments, an algorithm or set of algorithms transform data (e.g., epigenetic data, a database) into identifiable features of a bacteria or bacterial population. Algorithms utilized in embodiments herein make improvements in the fields of biomedical screening, diagnostic applications, bioforensics, drug discovery, diagnostic development, epidemiology, etc. In certain embodiments, algorithms may be implemented for by software.

The present methods allow rapid and accurate characterization of bacterial agents. The methods leverage biomedical research in virulence, pathogenicity, drug resistance and epigenomic sequencing into systems and methods that provide unprecedented levels of information from the nucleic acid of a bacteria. Thus, the methods are useful in a wide variety of fields. For example, commercial uses of this technology include, biomedical screening, diagnostic applications, bioforensics, drug discovery, diagnostic development, epidemiology, etc.

In some embodiments, provided herein is the use of unique epigenetic data (e.g., epigenomic sequence, epigenomic signature, etc.) to identify bacteria or bacterial populations and/or to characterize specific feature of as much. For example, Epigenomic data (e.g., epigenomic sequence or signature) can be used to understand how these methylated regions result in differences between species, strains, substrains, populations, etc. In some embodiments, mechanisms of virulence, invasion, evolution, interactions with other microbes, antibiotic resistance, etc. are characterized/compared. Epigenetic signatures are also used to identify regions as targets for diagnostics, therapeutics, and research; and to identify targets for vaccine development, protein recognition mechanisms, basic research to understand evolutionary aspects of proteins, and how they are used among different applications.

Epigenetic data (e.g., epigenomic sequence and/or signature) obtained and analyzed using the systems and methods described herein find use in species, strain, substrain, and/or population attribution in forensic analyses. It is envisaged that these DNA signatures can be used for real-time specific detection and characterization of bacteria, the source of which may then be attributed by monitoring the sequence and/or epigenetic differences identified and/or organized by the systems and methods herein. Detailed analysis of sequences/signatures across species, strains, substrains, populations, etc. will identify: epigenetic-encoded virulence factors, mechanisms of resistance, vaccine candidates, modes of pathogenicity, etc.

In certain embodiments, methods herein find use in forensic analysis, and can be used identify the source of an outbreak or biothreat, authenticate a sample, separate nucleic acids in a sample that potentially has multiple sources, determine characteristics of the sample, etc. As one example, epigenetic data (e.g., epigenomic sequence and/or signature) may find use in confirming that the sample is biological and not synthetic in origin.

1. A method of characterizing a microorganism in a sample comprising:
(a) sequencing nucleic acid from the microorganism, wherein said sequencing results in an epigenomic signature of said microorganism;
(b) comparing the epigenomic signature to a reference; and
(c) identifying characteristics of said microorganism based on similarities and/or differences between the epigenomic signature of said microorganism and the reference.

2. The method of claim 1, wherein said reference correlates at least one microbial characteristic with a whole-genome microbial reference signature.

3. The method of claim 1, wherein said reference correlates at least one microbial characteristic with a sub-genomic microbial reference signature.

4. The method of claim 1, wherein the at least one microbial characteristic is selected from species, strain, sub-strain, serotype, virulence level, pathogenicity, origin, known geographical range, antibiotic resistance or sensitivity, and culture conditions.

5. The method of claim 2, wherein the epigenomic signature is an who epigenomic sequence.

6. The method of claim 2, wherein the reference is a database of microbial epigenetic signatures.

7. The method of claim 6, wherein the reference is a database of microbial epigenomic signatures.

8. The method of claim 6, wherein the reference is a database of microbial epigenetic sequences.

9. The method of claim 8, wherein the reference is a database of microbial epigenomic sequences.

10. The method of claim 6, wherein comparing the epigenomic signature to a reference comprises querying the database for epigenomic signature matches.

11. The method of claim 1, wherein comparing the epigenomic signature to a reference comprises querying the reference for sub-genomic epigenetic signature matches.

12. The method of claim 1, wherein the sequencing is performed by a non-amplification sequencing technique.

13. The method of claim 1, wherein the sequencing is performed by a single molecule sequencing technique.

14. The method of claim 1, wherein steps (b) and (c) comprise:
(i) sending the epigenomic signature of said microorganism to a third party to be characterized; and
(ii) receiving a report identifying characteristics of said microorganism.

15. The method of claim 14, wherein the sending are receiving are performed electronically.

16. A method of characterizing a microbial bioagent comprising:
(a) exposing (i) a single nucleic acid molecule from the bioagent and (ii) sequencing reagents to conditions that allow determination of the epigenetic sequence of the single nucleic acid molecule;
(b) comparing the epigenetic sequence of the single nucleic acid molecule or a representation thereof to a reference; and
(c) identifying characteristics of the microorganism based on similarities between epigenetic sequence of the single nucleic acid molecule or a representation thereof to a reference.

17. The method of claim 16, wherein the single nucleic acid molecule is a fragment of a whole genome nucleic acid from the microorganism.

18. The method of claim 17, further comprising a step prior to step (a) of fragmenting the whole-genome nucleic acid from the microorganism.

19. The method of claim 17, wherein step (a) is performed in parallel for multiple single nucleic acid molecules that are fragments of the whole-genome nucleic acid from the microorganism.

20. The method of claim 19, comprising comparing the epigenetic sequence or a representation thereof or each of the multiple single nucleic acid molecules to the reference.

21. The method of claim 20, comprising identifying characteristics of the microorganism based on similarities between the epigenetic sequences or representations thereof of any of the multiple single nucleic acid molecules and the reference.

22. The method of claim 19, wherein the multiple single nucleic acid molecules collectively comprise the entire whole-genome nucleic acid from the microorganism.

23. The method of claim 22, further comprising generating an epigenomic sequence or an epigenomic signature from the epigenetic sequences of the multiple single nucleic acid molecules that are fragments of the whole-genome nucleic acid from the microorganism.

24. The method of claim 23, further comprising comparing the epigenomic sequence or the epigenomic signature to the reference.

25. The method of claim 24, comprising identifying characteristics of the microorganism based on similarities between the epigenomic sequence or the epigenomic signature and the reference.

26. The method of claim 16, wherein the reference is a database of epigenetic data of multiple different microorganism.

27. The method of claim 26, wherein the reference is a database of microbial epigenetic sequences, epigenetic signatures, or other representations thereof.

28. The method of claim 26, wherein the reference is a database of microbial epigenomic sequences, epigenomic signatures, or other representations thereof.

29. The method of claim 26, wherein the multiple different microorganism are: different species, different serotypes, different strains, different substrains, and/or grown under different conditions.

30. The method of claim 26, wherein each entry of epigenetic data in the database is correlated or indexed to characteristics of the respective microorganism.

31. A method of responding to a microbial threat comprising:
(a) obtaining a sample comprising:
(i) a microorganism that is a source of the microbial threat, or
(ii) genomic nucleic acid from a microorganism that is a source of the microbial threat;
(b) determining an epigenomic sequence, epigenomic signature, or other representation thereof for the microorganism that is a source of the microbial threat;
(c) comparing the epigenomic sequence, epigenomic signature, or other representation thereof to a database of microbial epigenomic sequences, epigenomic signatures, or other representations thereof, wherein the microbial epigenomic sequences, epigenomic signatures, or other representations thereof are indexed to characteristics of the respective microorganism; and
(d) identifying at least one microbial characteristic of the microorganism that is a source of the microbial threat based on similarities or identities between:
(i) the epigenomic sequence, epigenomic signature, or other representation thereof for the microorganism that is a source of the microbial threat, and
(ii) one or more microbial epigenomic sequences, epigenomic signatures, or other representations thereof of the database; and
(e) responding to the microbial threat.

32. The method of claim 31, wherein the at least one microbial characteristic is selected from species, strain, sub-strain, serotype, virulence level, pathogenicity, origin, known geographical range, antibiotic resistance or sensitivity, and culture conditions.

33. The method of claim 31, wherein the microbial threat is a microbial infection of an individual subject.

34. The method of claim 33, wherein responding to the microbial threat comprises treating the individual subject with an appropriate treatment based upon one or more of the at least one microbial characteristics.

35. The method of claim 33, wherein responding to the microbial threat comprises alerting public health officials of the identification of subject infected with microorganism having one or more of the at least one microbial characteristics.

36. The method of claim 31, wherein the microbial threat is a microbial infection of an outbreak of microbial infections across a population.

37. The method of claim 36, wherein responding to the microbial threat comprises treating the infected subjects with an appropriate treatments based upon one or more of the at least one microbial characteristics.

38. The method of claim 36, wherein responding to the microbial threat comprises alerting public health officials of the identification of a population infected with microorganism having one or more of the at least one microbial characteristics.

39. The method of claim 31, wherein the microbial threat comprises actual or potential bioterrorism.

40. The method of claim 36, wherein responding to the microbial threat comprises reporting to public health officials, government officials, police, or military the identification of a microbial threat having one or more of the at least one microbial characteristics.

41. A system comprising:
(a) a computer readable medium or computer memory component comprising a database, wherein said database comprise at least two microbial epigenomic sequences or signatures, wherein the at least two microbial epigenomic sequences or signatures are each correlated or indexed to one or more microbial characteristics; and
(b) a processor configured to query, build, or organize said database.

42. The system of claim 41, wherein the one or more microbial characteristics are selected from species, strain, sub-strain, serotype, virulence level, pathogenicity, origin, known geographical range, antibiotic resistance or sensitivity, and culture conditions.

43. The system of claim 42, wherein each microbial characteristic is correlated or indexed to a sub-genomic sequence or signature within microbial epigenomic sequences or signature.

44. A method of characterizing a microorganism in a sample, comprising querying the database of claim 43 with a microbial epigenomic sequence or signature of the microorganism, wherein a match between the microbial epigenomic sequence or signature of the microorganism and a microbial epigenomic sequence or signature in the database identifies one or more microbial characteristics of the microorganism in the sample.

45. A method of characterizing a microorganism in a sample, comprising querying the database of claim 43 with a microbial epigenomic sequence or signature of the microorganism, wherein a match between a portion of the microbial epigenomic sequence or signature of the microorganism and a sub-genomic microbial epigenetic sequence or signature in the database identifies one or more microbial characteristics of the microorganism in the sample.

46. A system comprising:
(a) a sequencing module configured to perform massively-parallel, single-molecule sequencing reactions capable of detecting the epigenetic sequence of multiple nucleic acid molecules; and
(b) a database comprising microbial epigenomic sequences or signatures for a plurality of microorganism, wherein each of the microbial epigenomic sequences or signatures are correlated or indexed to one or more microbial characteristics.

47. The system of claim 46 wherein the sequencing module and the database are located at the same physical location.

48. The system of claim 47, wherein the sequencing module and the database are located at the same physical location, but are electronically connected such that data may be sent and received between the sequencing module and the database.

49. The system or method of one or claims 1-48, wherein said microorganism is a bacteria.

50. The system or method of one or claims 1-48, wherein said microorganism is a virus.
